(19) United States
(12) Patent Application Publication
(10) Pub. No.: US 2024/0424299 A1
(43) Pub. Date: Dec. 26, 2024

(54) Sacral Neuromodulation for Bowel and Sexual Functions

(71) Applicant: University of Pittsburgh - Of the Commonwealth System of Higher Education, Pittsburgh, PA (US)

(72) Inventor: Changfeng Tai, Wexford, PA (US)

(21) Appl. No.: 18/700,913

(22) PCT Filed: Aug. 11, 2022
(86) PCT No.: PCT/US2022/040037

§ 371 Date: Apr. 12, 2024

Related U.S. Application Data
(60) Provisional application No. 63/255,606, filed on Oct. 14, 2021.

(51) Int. Cl. A61N 1/36; A61N 1/05

(52) U.S. Cl. CPC A61N 1/36062; A61N 1/0551; A61N 1/36007; A61N 1/36107; A61N 1/36153; A61N 1/36157; A61N 1/36171; A61N 1/36178

(57) ABSTRACT

Provided herein is a method of inducing colon contractions/defecation or penile erection in a patient, including stimulating one or more sacral roots of the patient's spinal cord with a plurality of electrical pulses, wherein the electrical pulses are delivered at a frequency of from about 3 Hz to about 10 Hz or from about 10 Hz to about 80 Hz.

### CROSS-REFERENCE TO RELATED APPLICATIONS

This application claims the benefit of U.S. Provisional Patent Application No. 63/255,606, filed Oct. 14, 2021, which is incorporated herein by reference in its entirety.

### STATEMENT REGARDING FEDERALLY SPONSORED RESEARCH

This invention was made with government support under Grant No. N66001-20-C-4050 awarded by the Department of Defense/Defense Advanced Research Projects Agency. The government has certain rights in the invention.

### BACKGROUND OF THE INVENTION

### Field of the Invention

Provided herein is a method of stimulating the sacral spinal cord/roots and related devices, more specifically a method of modulating bowel and sexual function by stimulating the sacral spinal cord/roots, and devices for carrying out such methods.

### Description of Related Art

A number of conditions arise from disruption of normal physiological processes in the lower pelvis. Conditions, such as urinary incontinence, overactive bladder, urine retention and voiding dysfunction, detrusor sphincter dyssinergia, fecal incontinence, constipation, irritable bowel syndrome, sexual dysfunction in both men and women, premature ejaculation, decreased sexual sensation, anorgasmia, urethral pain, prostate pain, vulvodynia, anal pain, rectal pain, and bladder pain are among those conditions. Those conditions can result from neurological impairment or from other diseases or conditions, for example spinal cord injury or stroke, trauma, disease (e.g., multiple sclerosis), and/or congenital defects.

In the past, such conditions have been treated by sacral anterior root stimulation, which requires a major invasive spinal surgery to expose the sacral spinal roots for implantation of stimulation electrodes. Sacral posterior rhizotomy prevents the dyssynergic contraction of anal sphincter, but it also eliminates spinal reflex defecation and sexual function such as penile erection. Therefore, a need exists for a minimally invasive neuromodulatory approach that does not require invasive spinal surgery and sacral posterior rhizotomy.

### SUMMARY OF THE INVENTION

Provided herein is a method of inducing colon contractions and/or defecation in a patient, including stimulating one or more sacral roots of the patient's spinal cord and/or one or more sacral segments of the patient's spinal cord with a plurality of electrical pulses, wherein the electrical pulses are delivered at a frequency of from about 3 Hz to about 10 Hz.

Also provided herein is a method of inducing a penile erection in a patient, comprising stimulating one or more sacral roots of the patient's spinal cord and/or one or more sacral segments of the patient's spinal cord with a plurality of electrical pulses, wherein the electrical pulses are delivered at a frequency of from about 10 Hz to about 80 Hz.

Further non-limiting embodiments are set forth in the following clauses:

Clause 1. A method of inducing colon contractions and/or defecation in a patient, comprising stimulating one or more sacral roots of the patient's spinal cord and/or one or more sacral segments of the patient's spinal cord with a plurality of electrical pulses, wherein the electrical pulses are delivered at a frequency of from about 3 Hz to about 10 Hz.

Clause 2. The method of clause 1, wherein the one or more sacral roots or sacral cord segments are one or more of the patient's S1, S2, S3, S4, and/or S5 sacral roots or sacral cord segments.

Clause 3. The method of clause 1 or 2, wherein the patient is human.

Clause 4. The method of any of clauses 1-3, wherein the one or more sacral roots or sacral cord segments of the patient's spinal cord innervate the patient's colon and rectum.

Clause 5. The method of any of clauses 1-4, wherein the stimulation is applied to the ventral/anterior and/or dorsal/posterior sacral roots.

Clause 6. The method of any of clauses 1-5, wherein the stimulation is applied to the patient's S2 and/or S3 sacral roots and/or sacral cord segments.

Clause 7. The method of clause 6, wherein the stimulation is applied to the patient's S2 and/or S3 ventral/anterior root.

Clause 8. The method of any of clauses 1-7, wherein the stimulation is applied at a frequency of about 7 Hz.

Clause 9. The method of any of clauses 1-8, wherein the stimulation is applied at an intensity that can induce colon/rectum contraction ranging about 0.1V to about 20 V and/or about 0.1 mA to about 20 mA.

Clause 10. The method of clause 9, wherein the stimulation is applied at an intensity of about 1 V and/or 1 mA.

Clause 11. The method of clause 9, wherein the stimulation is applied at an intensity of about 4 V and/or 4 mA.

Clause 12. The method of clause 9, wherein the stimulation is applied at an intensity of about 6 V and/or 6 mA.

Clause 13. The method of any of clauses 1-12, wherein the stimulation is applied continuously or intermittently.

Clause 14. The method of clause 13, wherein the intermittent stimulation is applied for about 1 minute followed by about 1 minute where no stimulation is applied.

Clause 15. A method of inducing a penile erection in a patient, comprising stimulating one or more sacral roots of the patient's spinal cord and/or one or more sacral segments of the patient's spinal cord with a plurality of electrical pulses, wherein the electrical pulses are delivered at a frequency of from about 10 Hz to about 80 Hz.

Clause 16. The method of clause 15, wherein the one or more sacral roots or sacral cord segments are one or more of the S1, S2, S3, S4, and/or S5 sacral roots or sacral cord segments.

Clause 17. The method of clause 15, wherein the patient is human.

Clause 18. The method of any of clauses 15-17, wherein the one or more sacral roots or sacral cord segments of the patient's spinal cord innervate the patient's penis.

Clause 19. The method of any of clauses 15-18, wherein the stimulation is applied to the ventral/anterior and/or dorsal/posterior sacral roots.

Clause 20. The method of any of clauses 15-19, wherein the stimulation is applied to the S1 and/or S2 sacral roots and/or sacral cord segments.

Clause 21. The method of clause 20, wherein the stimulation is applied to the S1 and/or S2 ventral/anterior root.

Clause 22. The method of any of clauses 15-21, wherein the stimulation is applied at a frequency of about 30 Hz to about 40 Hz.

Clause 23. The method of any of clauses 15-22, wherein the stimulation is applied at an intensity that can induce penile erection ranging about 0.1 V to about 20 V and/or about 0.1 mA to about 20 mA.

Clause 24. The method of clause 23, wherein the stimulation is applied at an intensity of about 3 V and/or 3 mA.

Clause 25. The method of clause 23, wherein the stimulation is applied at an intensity of about 6 V and/or 6 mA.

Clause 26. The method of any of clauses 15-25, wherein the stimulation is applied continuously or intermittently.

Clause 27. The method of any of clauses 15-26, wherein the stimulation causes an increase in pressure in the patient's corpus cavernosum of at least 50 cm H₂O.

Clause 28. The method of any of clauses 15-27, wherein the stimulation causes an increase in pressure in the patient's corpus cavernosum of at least 100 cm H₂O.

Clause 29. A system for inducing colon contractions and/or defecation in a patient, comprising: at least one lead configured to be placed in proximity to one or more sacral roots of the patient's spinal cord and/or one or more sacral segments of the patient's spinal cord; a pulse generator in electrical communication with the at least one lead; and at least one processor in communication with the pulse generator, wherein the processor is programmed or configured to cause the pulse generator to deliver one or more electrical pulses through the at least one lead at a frequency of from about 3 Hz to about 10 Hz.

Clause 30. The system of clause 29, wherein the processor is programmed or configured to cause the pulse generator to deliver one or more electrical pulses through the at least one lead at a frequency of about 7 Hz.

Clause 31. The system of clause 29 or clause 30 wherein the processor is programmed or configured to cause the pulse generator to deliver one or more electrical pulses through the at least one lead at an intensity that can induce colon/rectum contraction ranging about 0.1V to about 20 V and/or about 0.1 mA to about 20 mA.

Clause 32. The system of any of clauses 29-31, wherein the processor is programmed or configured to cause the pulse generator to deliver one or more electrical pulses through the at least one lead at an intensity of about 1 V and/or 1 mA.

Clause 33. The system of any of clauses 29-32, wherein the processor is programmed or configured to cause the pulse generator to deliver one or more electrical pulses through the at least one lead at an intensity of about 4 V and/or 4 mA.

Clause 34. The system of any of clauses 29-33, wherein the processor is programmed or configured to cause the pulse generator to deliver one or more electrical pulses through the at least one lead at an intensity of about 6 V and/or 6 mA.

Clause 35. The system of any of clauses 29-34, wherein the processor is programmed or configured to cause the pulse generator to deliver one or more electrical pulses through the at least one lead continuously or intermittently.

Clause 36. The system of any of clauses 29-35, wherein the processor is programmed or configured to cause the pulse generator to deliver one or more electrical pulses through the at least one lead for about 1 minute followed by about 1 minute where no stimulation is applied.

Clause 37. A system for inducing a penile erection in a patient, comprising: at least one lead configured to be placed in proximity to one or more sacral roots of the patient's spinal cord and/or one or more sacral segments of the patient's spinal cord; a pulse generator in electrical communication with the at least one lead; and at least one processor in communication with the pulse generator, wherein the processor is programmed or configured to cause the pulse generator to deliver one or more electrical pulses through the at least one lead at a frequency of from about 10 Hz to about 80 Hz.

Clause 38. The system of clause 37, wherein the processor is programmed or configured to cause the pulse generator to deliver one or more electrical pulses through the at least one lead at a frequency of about 30 Hz to about 40 Hz.

Clause 39. The system of clause 37 or 38, wherein the processor is programmed or configured to cause the pulse generator to deliver one or more electrical pulses through the at least one lead at an intensity that can induce penile erection ranging about 0.1 V to about 20 V and/or about 0.1 mA to about 20 mA.

Clause 40. The system of any of clauses 37-39, wherein the processor is programmed or configured to cause the pulse generator to deliver one or more electrical pulses through the at least one lead at an intensity of about 3 V and/or 3 mA.

Clause 41. The system of any of clauses 37-40, wherein the processor is programmed or configured to cause the pulse generator to deliver one or more electrical pulses through the at least one lead at an intensity of about 6 V and/or 6 mA.

Clause 42. The system of any of clauses 37-41, wherein the processor is programmed or configured to cause the pulse generator to cause the pulse generator to deliver one or more electrical pulses through the at least one lead continuously or intermittently.

### BRIEF DESCRIPTION OF THE DRAWINGS

FIGS. 1A-1C are schematic diagrams of various aspects of external systems (FIGS. 1A and 1B), and implantable systems (FIG. 1C) for use in stimulating the spinal roots and/or the spinal cord as described herein.

FIG. 2 shows an experimental setup for electrical stimulation of sacral roots S1, S2, or S3 to induce colon contractions according to non-limiting embodiments described herein.

FIG. 3 shows distal and proximal colon responses to stimulation of sacral S1-S3 ventral roots at different frequencies (1-50 Hz). Panel A. S1 ventral root. Panel B. S2 ventral root. Panel C. S3 ventral root. Data were obtained from the same animal.

FIGS. 4A-4B shows distal and proximal colon responses to sacral ventral root stimulation are frequency and spinal segment dependent. FIG. 4A, Panel A. S1 ventral root. FIG. 4A, Panel B. S2 ventral root. FIG. 4B. S3 ventral root. The contraction amplitude induced by different stimulation frequencies in different sacral segments is normalized to the maximal response in the same animal. Stimulation intensity (4-16 V) for S2 ventral root was 1.5-3 times threshold intensity for 7 Hz stimulation to induce an observable contraction of proximal colon. When S1 and S3 ventral root stimulation did not induce a colon contraction, the maximal stimulation intensity (8-12 V) without causing lower body movement was used to test different frequencies. *=significantly different from 1 Hz (one-way ANOVA); #=significantly different from S2 ventral root (two-way ANOVA). N=7 cats.

FIG. 5 shows distal and proximal colon responses to intermittent (5×1 minute) or long duration (5 minutes) continuous stimulation of both S2 ventral and dorsal roots. Panel A. Pressure traces showing distal and proximal colon contractions. The intensity threshold (T) for proximal colon contraction was 4 V, and 6 V (1.5T) was used to induce the intermittent or continuous contractions. Panel B. Summarized results (N=4 cats) showing that the contraction amplitudes were maintained during intermittent stimulation (7 Hz, 0.2 ms, 1.5-4T, T=2-4 V). Panel C. Summarized results (N=7 cats) showing that the maximal (Max) contraction amplitude was significantly reduced from mean 30 cmH₂O to about 18 cmH₂O at the end of the continuous 5-minute stimulation (7 Hz, 0.2 ms, 1.5-3T, T=2-4 V). *=significantly different (p<0.05, paired t-test).

FIG. 6 shows defecation induced by stimulation of S2 ventral and dorsal roots. Panel A. In cat #1 after inserting three marbles into the rectum, the defecation induced by sacral S2 root stimulation eliminated 2 marbles after 1 minute and 2 minutes of stimulation, respectively, with the last marble partially evacuated during the last 2 minutes of the 6-minute stimulation. Panel B. In cat #2 after inserting 1 marble into the rectum, it was defecated after 2.5 minutes of stimulation. After inserting 4 marbles, stimulation of 11 minutes produced defecation of 1 marble at 4.5 minutes with 3 residual marbles in the rectum. Colon contraction pressure was measured by a large condom catheter with the condom covering the proximal and distal colon.

FIG. 7 shows an experimental setup for electrical stimulation of sacral roots S1, S2, or S3 to induce penile erection according to non-limiting embodiments described herein.

FIG. 8 shows penile pressure in the corpus cavernosum induced by electrical stimulation of sacral ventral roots at different frequency and different spinal segment. Panel A. In a cat, S1 ventral root stimulation (10-80 Hz) of short duration (1 minute) induced a large increase (200 cmH₂O) in penile pressure lasting several minutes even after termination of the stimulation. Panel B. In another cat, S2 ventral root stimulation (30-80 Hz) of short duration (1 minute) was most effective in inducing large increases (150 cmH₂O) in penile pressure. Panel C. Spinal segmental effect on penile pressure induced by electrical stimulation of the ventral roots. *=significantly different from S1 ventral root (p<0.05, ANOVA). Stimulation: 30 Hz, 0.6-12 V, 0.2 ms. Panel D. Frequency effect on penile pressure induced by stimulation of S1 or S2 ventral root. *=significantly different from 5 Hz (p<0.05, ANOVA). Stimulation: 0.6-12 V, 0.2 ms.

FIG. 9 shows penile pressure in the corpus cavernosum induced by prolonged (10 minutes) electrical stimulation of sacral spinal root (both ventral and dorsal roots). Panel A. In a cat, S1 spinal root stimulation induced a large increase (160 cmH₂O) in penile pressure that was maintained during the entire 10 minutes of stimulation. Panel B. In another cat, S2 spinal root stimulation gradually increased the penile pressure during the first 9 minutes and then triggered a fast increase in pressure during the last 1 minute of stimulation reaching a penile pressure of 140 cmH₂O at the end of the stimulation. Panel C. On average (N=8 cats) the penile pressure at the end of 10-minute stimulation was maintained at the same level of the maximal pressure generated during the stimulation.

FIG. 10 shows penile pressure in the corpus cavernosum induced by prolonged (10 minutes) electrical stimulation of sacral spinal root (both ventral and dorsal roots) before and after a complete spinal cord transection at the T9-T10 level. Panel A. Before spinal transection, S1 spinal root stimulation in a cat induced a maximal 175 cmH₂O increase in penile pressure and then maintained a high pressure during the 10 minutes of stimulation. Panel B. In the same cat, 10 minutes after spinal cord transection S1 spinal root stimulation induced a small (30 cmH₂O) increase in penile pressure during the first 3 minutes and then triggered a fast pressure increase reaching a maximal penile pressure of 150 cmH₂O. Panel C. On average (N=6 cats) the maximal penile pressure induced by sacral S1/S2 root stimulation was significantly reduced by the acute spinal cord transection. *=a significant difference (p=0.0025, paired t-test).

### DESCRIPTION OF THE INVENTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates, and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

As used herein, the term “comprising” and like terms are open-ended. The term “consisting essentially of” limits the scope of a claim to the specified materials or steps and those that do not materially affect the basic and novel characteristics of the claimed invention. The term “consisting of” excludes any element, step, or ingredient not specified in the claim.

As used herein, the terms “a” and “an” refer to one or more.

As used herein, the term “patient” is any mammal, including humans, and a “human patient” is any human

As used herein, the terms “communication” and “communicate” refer to the receipt, transmission, or transfer of one or more signals, messages, commands, or other type of data. For one unit or device to be in communication with another unit or device means that the one unit or device is able to receive data from and/or transmit data to the other unit or device. A communication can use a direct or indirect connection, and can be wired and/or wireless in nature. Additionally, two units or devices can be in communication with each other even though the data transmitted can be modified, processed, routed, etc., between the first and second unit or device. For example, a first unit can be in communication with a second unit even though the first unit passively receives data and does not actively transmit data to the second unit. As another example, a first unit can be in communication with a second unit if an intermediary' unit processes data from one unit and transmits processed data to the second unit. It will be appreciated that numerous other arrangements are possible. Any known electronic communication protocols and or algorithms can be used such as, for example, TCP/IP (including HTTP and other protocols), WLAN (including 802.1 la/b/g/n and other radio frequency-based protocols and methods), analog transmissions, Global System for Mobile Communications (GSM), 3G/4G/LTE, BLUETOOTH, ZigBee, EnOcean, TransferJet, Wireless USB, and the like known to those of skill in the art.

As used herein, “electrical communication,” for example in the context of transmitting electrical pulses from a pulse generator to an electrode refers to sending an electrical pulse produced by a pulse generator to a skin surface electrode, an electrode lead, a magnetic coil, or like devices capable of generating electrical current to stimulate a nerve or neuron as described herein, typically through an electrically-conductive lead, such as a wire.

As used herein, the “intensity threshold” (T) means the minimal intensity that can induce a desired physiological response such as colon/rectum contraction, defecation, or penile erection and/or the minimal intensity that can induce a contraction in the proximal portion of the colon, and/or an increase in pressure in the colon or the corpus cavernosum.

The “intensity” of an electrical pulse is proportional to, and refers to the voltage (V) and/or current (e.g., milliAmperes or mA) applied to the nerve or neuron, with an increased intensity being proportional to an increased voltage or an increased current applied to the nerve or neuron. Those of skill will appreciate that, assuming electrode-tissue resistance of 1 kOhm, 1 V is approximately equal to 1 mV in terms of intensity.

U.S. Pat. No. 8,805,510 is incorporated by reference herein in its entirety.

Provided herein are methods, and devices/systems useful in carrying out such methods, of stimulating the sacral spinal cord/roots of a patient to elicit a physiological response, in particular colon/rectum contraction and/or defecation. Also provided herein are methods, and devices/systems useful in carrying out such methods, of stimulating the sacral spinal cord/roots of a patient to elicit a physiological response, in particular a penile erection. Useful stimulation can be electrical, through an implanted pulse generator, or non-invasive, through transcutaneous methods, such as transcutaneous electrical stimulation. Useful stimulation may also be non-invasive, through a magnetic stimulator that can be placed near or applied to an outer surface of the body to induce electrical current in the body to stimulate the spinal roots/cord, for example by using a conductive coil outside the body to generate a magnetic field for inducing an electrical current at the target(s) of interest. The methods and devices/systems disclosed herein are superior to other methods/systems, for example stimulation of the ventral root (e.g., in animals), at least because such other methods require a more invasive electrode placement.

The electrical stimulation described herein can include electrical pulses that can have any suitable characteristic, so long as the stimulation is effective to achieve the desired physiological response. As such, the terms “electrical stimulation” and “electrical pulses” are used interchangeably herein. As will be recognized by a person of skill in the art, characteristics of the electrical pulses, including, without limitation, amplitude (pulse strength, referring to the magnitude or size of a signal voltage or current), voltage, amperage, duration (e.g., pulsewidth), frequency, polarity, phase, relative timing, and symmetry of positive and negative pulses in biphasic stimulation, and/or wave shape (e.g., square, sine, triangle, sawtooth, or variations or combinations thereof) may be varied in order to provide the desired physiological response. So long as other characteristics of the electrical signals (e.g., without limitation, amplitude, voltage, amperage, duration, polarity, phase, relative timing and symmetry of positive and negative pulses in biphasic stimulation, and/or wave shape) are within useful ranges, modulation of the pulse frequency will achieve the desired physiological response.

One characteristic of the electrical signals used to produce a desired response, as described above, is the frequency of the electrical pulse. Although effective ranges (e.g., frequencies able to produce a stated effect) may vary from subject-to-subject, and the controlling factor is achieving a desired outcome, certain, non-limiting exemplary ranges may be as follows. In non-limiting embodiments, where the desired physiological response is defecation (e.g., colon contractions), the frequency does not exceed 10 Hz. In non-limiting embodiments or aspects, the stimulation is delivered at a frequency of about 1 Hz to about 10 Hz, about 3 Hz-10 Hz, about 5 Hz to about 10 Hz, about 7 Hz-10 Hz, about 5 Hz, about 7 Hz, or any subrange or value therebetween. In non-limiting embodiments, where the desired physiological response is a penile erection, useful frequencies range from about 10 Hz to about 80 Hz, optionally about 20 Hz to about 70 Hz, optionally about 20 Hz to about 50 Hz, optionally about 30 Hz to about 50 Hz, optionally about 30 Hz to about 40 Hz, optionally about 30 Hz, optionally about 40 Hz, all values and subranges therebetween inclusive. In non-limiting embodiments, the electrical pulses are delivered with a pulse width of about 0.2 ms.

As indicated above, a characteristic of electrical pulses is their intensity which in a medium of stable or relatively stable resistance, such as mammalian tissue, can be characterized as relating to current (I, typically measured in mA), or voltage (V, typically measured in mV or V), based on Ohm's Law. It should, therefore, be understood that the intensity of the stimulation is a matter of both V and I, and as such, both are increased, e.g., proportionally or substantially proportionally, with increased intensity of stimulation. As such, one characteristic of the pulses is the current that is applied to produce a physiological response. Stimulation can be achieved in a typical range of from 0.01 mA to 20 mA and/or 0.1 V to 20 V, all subranges and values therebetween inclusive.

Another characteristic of the intensity of the pulses is voltage. Stimulation can be achieved in a typical range of from 1 mV to 20 V, all subranges and values therebetween inclusive. In non-limiting embodiments or aspects, the stimulation is delivered with electrical pulses having a voltage of from about 0.8 V to about 16 V, about 2 V to about 16 V, about 4 V to about 16 V, about 6 V to about 16 V, about 0.9 V, about 1 V, about 2 V, about 3 V, about 4V, about 6 V, or any subrange or value therebetween.

As discussed above, the intensity threshold (T) of the electrical pulses delivered during stimulation can be defined as the minimal intensity that can induce a desired physiological response such as colon/rectum contraction, defecation, or penile erection and/or the minimal intensity that can induce a contraction in the proximal portion of the colon, and/or an increase in pressure in the colon or the corpus cavernosum. Useful ranges in terms of the intensity of the electrical pulses that can be delivered, relative to the threshold, can include about IT to about 4T, about 1.5T to about 3T, about IT, about 1.5T, about 3T, about 4T, or any subrange or value therebetween. In non-limiting embodiments, the intensity of the electrical pulses is 1.5T or 2T or 2.5 T or 3T.

As indicated above, the waveform of the pulses may vary, so long as the desired physiological response is realized. One skilled in the art will appreciate that other types of electrical stimulation may also be used in accordance with the present invention. Monophasic or biphasic stimuli, or a mixture thereof, may be used. Damage to nerves by the application of an electrical current may be minimized, as is known in the art, by application of biphasic pulses or biphasic waveforms to the nerve(s), as opposed to monophasic pulses or waveforms that can damage nerves in some instances of long-term use. “Biphasic current,” “biphasic pulses,” or “biphasic waveforms” refer to two or more pulses that are of opposite polarity that may be of equal or substantially equal net charge (hence, biphasic and charge balanced), and may be symmetrical, asymmetrical, or substantially symmetrical. This is accomplished, for example, by applying through an electrode one or more positive pulses, followed by one or more negative pulses, typically of the same amplitude and duration as the positive pulses, or vice versa, such that the net charge applied to the target of the electrode is zero, or approximately zero. For charge-balanced biphasic stimulation, the opposite polarity pulses may have different amplitudes, profiles, or durations, so long as the net applied charge by the biphasic pulse pair (the combination of the positive and negative pulses) is approximately zero.

The waveform may be of any useful shape, including without limitation: sine, square, rectangular, triangular, sawtooth, rectilinear, pulse, exponential, truncated exponential, or damped sinusoidal. The pulses may increase or decrease over the stimulation period. In aspects, the waveform is rectangular. The pulses may be applied continuously or intermittently as needed. For example, the stimulation may be applied for short intervals (e.g., 1-10 minutes) or longer intervals (360 minutes or even longer, for example days, weeks, months, or even years) to achieve longer-lasting physiological responses, in terms of hours, days, weeks, months, or years. In aspects, the stimulation is applied for at least 5 minutes. In non-limiting embodiments, the stimulation is applied for about 1 minute, at 1 minute intervals (e.g., 1 minute of stimulation, followed by 1 minute of no stimulation). In non-limiting embodiments, the stimulation is delivered until 5 minutes of total stimulation is delivered. In non-limiting embodiments, intermittent stimulation is followed a period of continuous stimulation. In non-limiting embodiments or aspects, the stimulation is delivered only when the physiological response is desired.

As described above, the stimulation may be applied intermittently (that is, the pulses are turned on and off alternately during a stimulation interval for any time period) during continuous or interval stimulation protocols. For example, the stimulation may be applied for 5 seconds on and 5 seconds off over an interval of, for example, 1-10 minutes or longer (e.g., hours, days, weeks, months, years). Other examples of intermittent application of pulses may be 1-90 seconds on and 1-90 seconds off over up to a 360 minute time period. For example, intermittent application of pulses may be continuous, that is, for as long as the pulses are having the desired effect, and for as long as the patient desires (i.e., is not painful or harmful to the patient). In one aspect, the stimulation is provided continuously, for example, to treat severe symptoms, or any symptom that does not respond to intermittent, short-term stimulation to the degree desired by a clinician or the patient.

Stimulation as described herein can be applied to the sacral roots of a patient's spinal cord and/or sacral spinal cord to produce a desired physiological response. In non-limiting embodiments, the desired physiological response is colon/rectum contraction and/or defecation. In non-limiting embodiments, the desired physiological response is a penile erection. In non-limiting embodiments or aspects, the patient has a spinal cord injury, or is suffering from a condition, such as constipation, optionally chronic constipation. Without wishing to be bound by the theory, constipation, such as chronic constipation, is relieved by the methods described herein by stimulating contraction of the colon/rectum. In non-limiting embodiments or aspects, the S1, S2, S3, S4, and/or S5 sacral roots and/or cords are stimulated with pulses having parameters as described herein. Those of skill in the art will appreciate that the specific sacral root(s) and/or sacral spinal cord segments that are stimulated will depend on the physiological response that is desired, as well as the identity of the patient, specifically the species of the patient. For example, in the Examples provided below, cats are used as experimental subjects, and those of skill appreciate that the feline sacral roots may differ from those of a human. See, e.g., Toossi et al., Comparative neuroanatomy of the lumbosacral spinal cord of the rat, cat, pig, monkey, and human. Scientific Reports, 2021, 11(1955). Thus, while a physiological response may be produced in a cat by stimulating the S1, S2, and/or S3 ventral root, dorsal root, or both, those of skill will appreciate that the human equivalent will be identified by different terminology (e.g., anterior root, posterior root, or both), and may involve stimulation of different roots, as the human spinal cord includes S4 and S5 sacral roots, where the feline spinal cord does not. Moreover, the S1 root of the cat may correspond to a different root in a human. Those of skill will appreciate that the key is targeting one or more sacral roots that innervate the target tissue/organ of interest (e.g., the colon or the penis).

In non-limiting embodiments the desired physiological response is defecation (e.g., colon contraction), the patient is a human, and the S1, S2, S3, S4, and/or S5 anterior roots are stimulated. In non-limiting embodiments, only the S2 root is stimulated, optionally both the ventral/anterior and dorsal/posterior roots, optionally only the ventral/anterior S2 root. In non-limiting embodiments, the patient is a human and the S1-S3 ventral/anterior and dorsal/posterior roots are stimulated.

In non-limiting embodiments the desired physiological response is a penile erection, the patient is a human, and the S1, S2, S3, S4, and/or S5 anterior roots are stimulated. In non-limiting embodiments, only the S1 root is stimulated, optionally both the ventral/anterior and dorsal/posterior roots, optionally only the ventral/anterior S2 root. In non-limiting embodiments, the patient is a human and the S1 and S2 roots are stimulated, optionally both the ventral/anterior and dorsal/posterior roots In non-limiting embodiments, the patient is a human and the S1-S3 ventral/anterior and dorsal/posterior roots are stimulated. In non-limiting embodiments, the physiological outcome is measured by an increase in pressure, or as an obtained pressure, within the corpus cavernosum of the penis. In non-limiting embodiments or aspects, the stimulation causes increase of at least 25, at least 50, at least 100, at least 125, and/or at least 150 cm H₂O within the patient's corpus cavernosum, all values and subranges therebetween inclusive. In non-limiting embodiments or aspects, the stimulation causes pressure within the patient's corpus cavernosum to reach at least 100, at least 125, and/or at least 150 cm H₂O, all values and subranges therebetween inclusive.

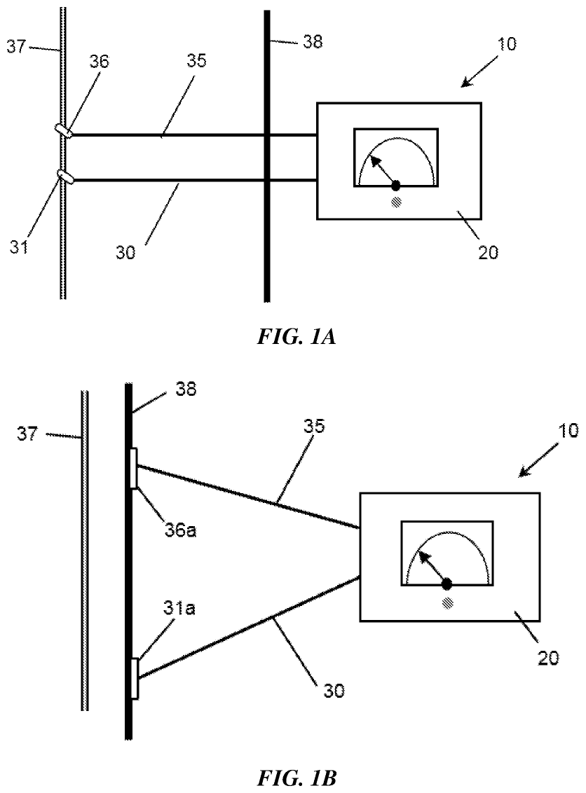

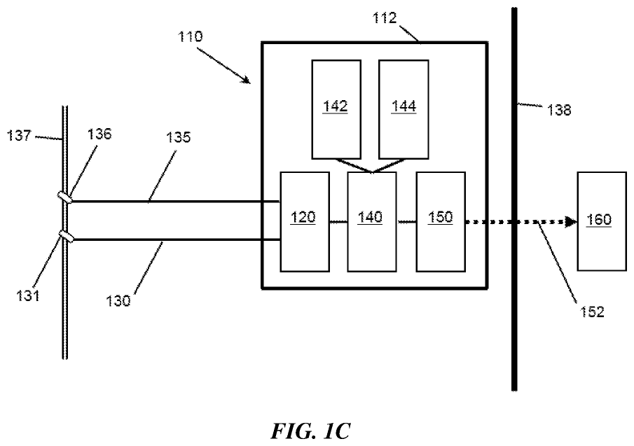

Turning to the figures, also provided herein are devices for applying stimulation with parameters as described herein, in a manner sufficient to induce the desired physiological response. FIG. 1A provides a general schematic of one non-limiting embodiment or aspect of an electrical stimulation device 10 useful in aspects of the methods described herein. The device 10 includes a power supply or pulse generator 20. The power supply/pulse generator 20 may be fixed output, or may be adjustable, for example within a useful range as described herein. The device 10 includes a first conductive lead 30 and a first nerve cuff 31, and a second conductive lead 35 with a second nerve cuff 36. The conductive leads 30 and 35 can be combined into a single lead to connect the nerve cuffs 31 and 36. In embodiments (not shown), the nerve cuffs 31 and 36 can also be combined into a single cuff, or they can be completely eliminated and replaced by conductive metals/electrodes located on the conductive leads 30 and 35 or located on the single lead that combines both 30 and 35. Conductive leads 30, 35 can be directly wired to power supply/pulse generator 20, or may each comprise multiple leads and electrical connectors, fasteners, terminals, or clips to produce a contiguous electrical connection between the power supply/pulse generator 20 and the respective nerve cuffs. A nerve 37 also is depicted. Skin 38 is also shown, and as such the device 10 is external and can be a hand-held or body-worn device-held in place by a belt or strap, such as by a hook and loop fastener band, though in aspects, the device 10 can be an implantable device (described in more detail below). In FIG. 1A, the leads are of opposite polarity and, together, form a circuit for application of any electrical waveform described herein. Alternative designs, with different leads, probes, electrodes, or electrical contacts, or combinations thereof will be apparent to those of ordinary skill. As used herein, an “electrical contact” is inclusive of any structure useful for directly applying an electrical current to a nerve or tissue in a patient, such as to the skin of a patient. Structures for producing a magnetic field, and, therefore, an electrical current via induction, are not considered to be electrical contacts. Nevertheless, in aspects, induction probes, that is structures capable of generating a magnetic field capable of producing an electrical current, may be used to produce the electrical pulses described herein.

FIG. 1B depicts schematically another aspect of a device 10 for stimulation, which, like the device of FIG. 1A, has an external power supply. In FIG. 1B, like reference numbers as compared to reference numbers of FIG. 1A, refer to like elements of the device 10. However, surface electrodes 31a and 36a replace nerve cuffs 31 and 36 of FIG. 1A, and stimulation is transcutaneous. In an alternative aspect, not shown, surface electrodes 31a and 36a are replaced by electromagnets for magnetic induction stimulation of impulses in nerve 37.

FIG. 1C depicts a further aspect of the nerve stimulation device 110 that is implanted, and includes an implantable housing 112. Commercial, implantable stimulators are known to those of skill in the art, for example from Medtronic (Dublin, IE) and can be useful for the purposes set forth in the present application, so long as they can be programmed to deliver stimulation as set forth herein. The housing 112 contains various subunits of the device, including a power supply/pulse generator 120 connected to a first lead 130 connected to a first nerve cuff 131, and a second lead 135 connected to a second cuff 136 for stimulating a nerve 137. Skin 138 is depicted for context. The conductive leads 130 and 135 can be combined into a single lead to connect the nerve cuffs 131 and 136. As described above, in aspects (not shown), the nerve cuffs 131 and 136 can also be combined into a single cuff, or they can be completely eliminated and replaced by conductive metals/electrodes located on the lead 130 and 135 or located on the single lead that combines both 130 and 135. For monopolar stimulation, one of the cuff/electrode can be located on the housing 112. The housing may be composed of any biocompatible material as are known in the medical fields for use in such implantable devices, such as a plastic, metal, carbon fiber, or ceramic material, or a polymer-coated material, such as a metal or plastic housing coated with a biocompatible polymer or hydrogel. The housing 112 also contains various connected subunits of the device 110, including a processor 140, a storage module 142 including transient data storage (e.g., RAM), and non-transient data storage, such as flash memory or a solid-state drive, and a battery 144 that is optionally rechargeable by magnetic induction. The processor 140 can also be connected to a wireless communications module 150 for communicating wirelessly, e.g., by near field communication, or by BLUETOOTH, ZigBee, Z-wave, Wi-Fi, or over a cellular network, with an external computer or computer network, such as a smartphone, tablet, laptop, personal computer, smart watch, workstation, server, or computer network.

The devices of FIGS. 1A-1C can be battery-powered, and optionally the battery is rechargeable. Where the device is implanted, the device can be recharged by wireless, e.g., magnetic induction recharging methods, as are known. The devices of FIGS. 1A-1C also can include a communications interface, such as a wireless communications interface or module, for transmitting data, and for receiving instructions from a separate computing device, such as from a controller app or software on a smartphone, tablet, laptop, personal computer, workstation, server, or computer network. As would be appreciated by those of ordinary skill in the fields of computer and software engineering, a multitude of potential device and system configurations and implementation schemes can be used to control devices and systems that provide electrical stimulation as described herein.

Referring to FIG. 1C, but equally applicable to any aspect of the device, e.g., device 10 of FIG. 1A and/or FIG. 1B, the device 110 comprises a controller for executing functions related to electrical pulse output of the power supply. In some examples, a controller is a central processing engine including a baseline processor, memory, and communications capabilities. For example, the controller can be any suitable processor comprising computer readable memory and configured to execute instructions either stored on the memory or received from other sources. Computer readable memory can be, for example, a disk drive, a solid-state drive, an optical drive, a tape drive, flash memory (e.g., a non-volatile computer storage chip), cartridge drive, and control elements for loading new software.

In some examples, the controller includes a program, code, a set of instructions, or some combination thereof, executable by the processor for independently or collectively instructing the device to interact and operate as programmed, referred to herein as “programming instructions”. In some examples, the controller is configured to issue instructions to the power supply/pulse generator to initiate electrical pulses, and to control output parameters of the power supply in a manner sufficient to stimulate the sacral spinal cord/roots. Those of skill in the art will appreciate that a processor associated with a device 10, 110 disclosed herein can be programmed to deliver stimulation as described generally throughout this disclosure. In any case, the controller is configured to receive and process electrical pulse parameters, either programmed into the device or from an external source, and optionally to output data obtained from the power supply as feedback to determine if the power supply is producing a desired output. Processing can include applying filters and other techniques for removing signal artifacts, noise, baseline waveforms, or other items from captured signals to improve readability.

Further to the above, the device 10, 110 can include programming instructions that, when executed by the processor 140, cause the power supply/pulse generator 120 to apply electrical stimulation at an intensity to provide a desired physiological response as described herein. These parameters are described above, but can include stimulation at from 1 Hz to 80 Hz, at an intensity of 0.01 mA to 20 mA and/or from 0.01 V to 20 V, for a duration of seconds to minutes, hours, days, or continuously or intermittently, all subranges therebetween inclusive for all parameters.

### EXAMPLE 1

The experimental protocol and animal use in this study were approved by the Animal Care and Use Committee at the University of Pittsburgh.

### Materials and Methods

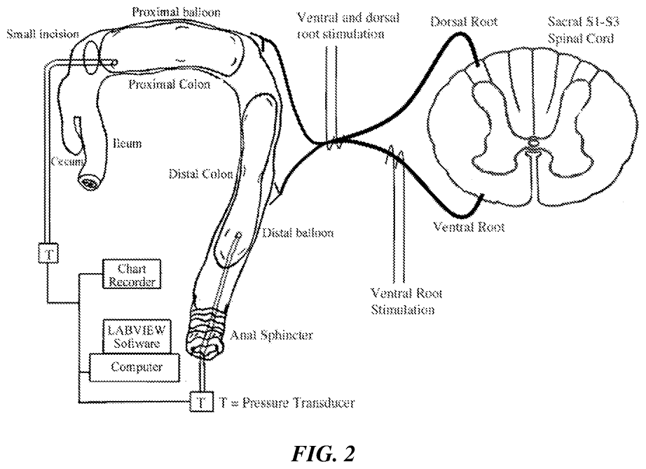

A total of 9 cats (4 females and 5 male, 4.0±0.3 kg; Liberty Research, Waverly, NY) were anesthetized with isoflurane (2-5% in oxygen) during surgery and then switched to α-chloralose anesthesia (initial 65 mg/kg i.v. and supplemented as needed) during data collection. Left cephalic vein was catheterized for fluid administration. A tracheotomy was performed, and a tube was inserted to keep the airway patent. A catheter was inserted into right carotid artery to monitor systemic blood pressure. Heart rate and blood oxygen were monitored by a pulse oximeter (9847V; NONIN Medical, Plymouth, MN) attached to the tongue. Through an abdominal incision, one balloon catheter (G15766, Cook Urological, Spencer, IN) 1.4 cm in diameter and 5 cm in length was inserted into the proximal colon via a small incision at the proximal end of the colon (FIG. 2). The second balloon catheter of the same size was inserted into the distal colon via the anus. The two balloons were filled with water (5-7 ml) to a resting pressure of 10-15 cmH₂O to measure proximal and distal colon contractions in the first group of 7 cats. In the second group of 2 cats, a large balloon catheter made of a condom (3.5 cm in diameter and 15 cm in length) was inserted via the small incision at the proximal end of the colon to occupy the entire colon so that both proximal and distal colon contractions were recorded. The condom was filled with water (60-70 ml) to a resting pressure of 10-15 cmH₂O. In these 2 cats, 1-4 glass marbles (diameter 1.5 cm) were also inserted into the rectum via the anus so that evacuation of these marbles could be videotaped simultaneously with the colon pressure recording.

The spinal cord was exposed from lumbar L7 to sacral S3 segments by a dorsal laminectomy. The dura mater was opened and each of the S1-S3 roots was identified. The dorsal and ventral roots were separated to either stimulate the ventral root individually or to stimulate both dorsal and ventral roots together. The animals were in a prone position during the experiment with a surgical retractor maintaining the spinal incision open to form a pool that was filled with warm (35-37° C.) mineral oil. A heating pad was used to maintain the animal body temperature between 35° C. and 37° C. Bipolar stainless steel hook electrodes (2-3 mm distance between the electrodes) were used to deliver the stimulation to each sacral spinal root by slightly lifting the root above the spinal cord.

In the first group of 7 cats, stimulation (7 Hz frequency, 0.2 ms pulse width, and 1 min duration) was delivered via the hook electrode to individual sacral ventral roots (S1, S2, or S3) at different intensity (1-16 V) to determine the intensity threshold (T) for inducing an observable pressure increase in the proximal colon. Then, an intensity (1.5-3T) that induced the maximal colon contraction was used to test colon responses to different stimulation frequencies (1-50 Hz). If no colon contraction was induced by stimulation of an individual ventral root, the maximal stimulation intensity that did not produce movement of the animal's lower body was used to test the frequency response. It was the study intention not to fix the hip and lower spine, so that sacral root stimulation by the hook electrode could produce local movement of the tail or hindlimb. However, the maximal stimulation intensity was limited by any movement of the lower spine, which would displace the hook electrode from the nerve. After stimulating each ventral root, the S2 ventral root which elicited the largest colon contraction was combined with the S2 dorsal root and the combination was stimulated with the most effective frequency (7 Hz.) The combined S2 root stimulation (1 minute duration) was applied 5 times at 1-minute intervals, and then followed by a continuous 5-minute stimulation to determine any fatigue in colon contraction.

In the second group of 2 cats, the combined S2 ventral and dorsal roots were stimulated (7 Hz) at an intensity that induced the largest colon contraction and for various durations (2.5-11 minutes) to determine if the stimulation could induce defecation, i.e., the evacuation of marbles that were inserted into the rectum. The defecation induced by S2 root stimulation was videotaped and the time of evacuation of the marbles was marked on the colon pressure recording.

To compare the colon contractions induced by stimulation of different ventral roots at different frequencies, the contraction amplitudes were always normalized to the maximal contraction amplitude induced by stimulation in the same animal. The colon contractions induced by the repeated 1-minute stimulations were normalized to the maximal contraction amplitude induced during the repeated simulations to determine repeatability. The maximal contraction amplitude induced during the 5-minute continuous stimulation and the contraction amplitude at the end of the 5-minute stimulation were compared to determine any fatigue during the continuous prolonged stimulation. The data from different animals are averaged and presented as means±SE. Statistical significance (p<0.05) was determined by paired t-test or repeated-measures ANOVA followed by Dunnett multiple comparison (one-way) or Bonferroni multiple comparison (two-way).

### Results

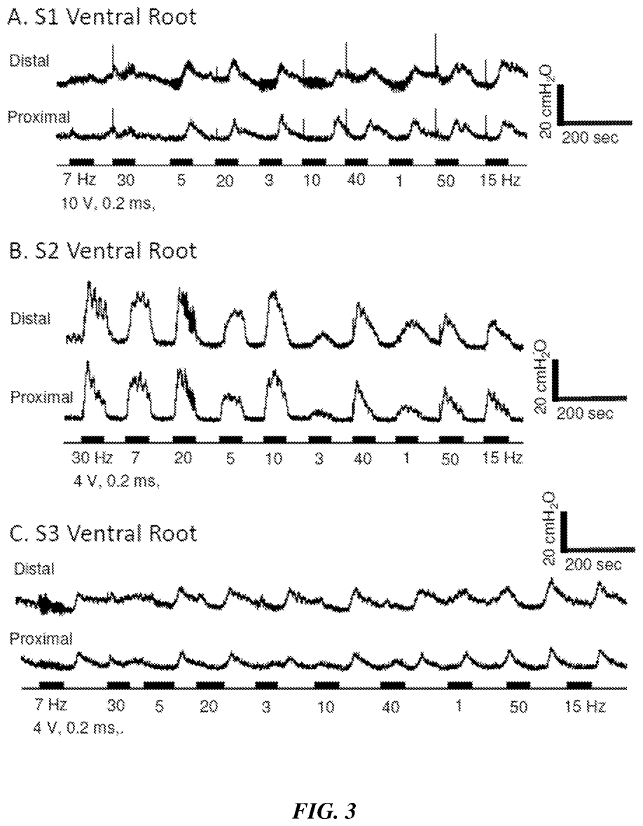

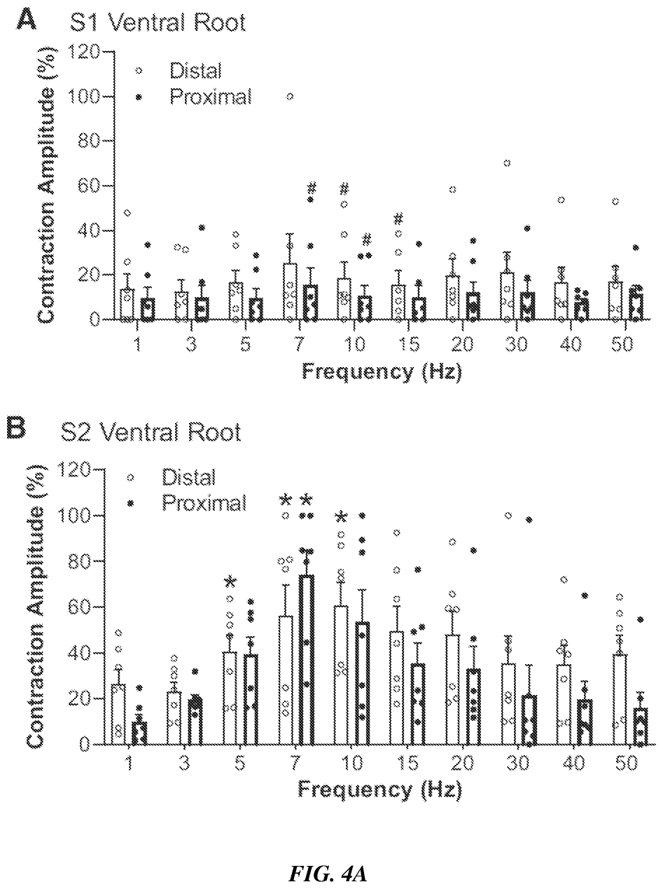

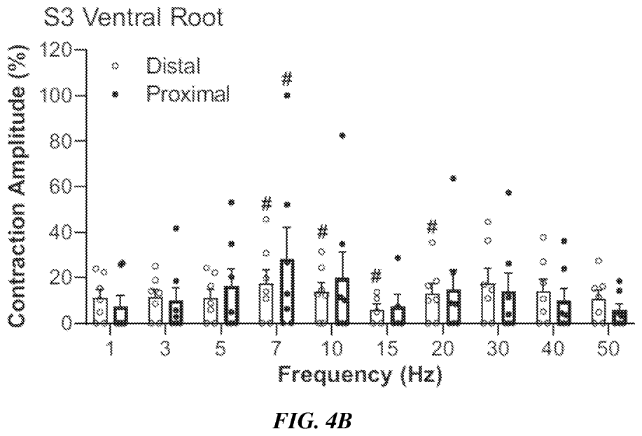

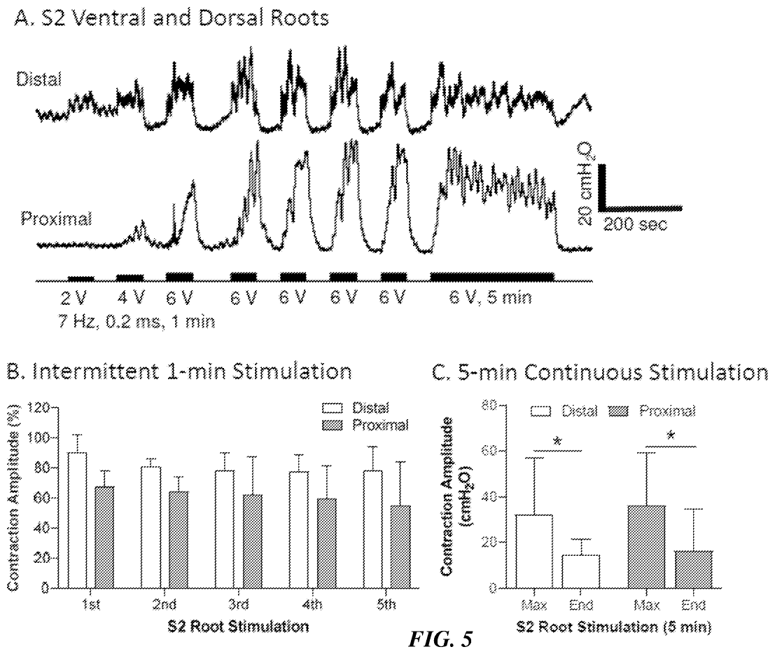

Stimulation of S2 ventral root induced larger colon contractions than stimulation of S1 or S3 ventral roots, and the most effective stimulation frequency was 7-10 Hz (FIGS. 3-4B). FIG. 3 shows the distal and proximal colon pressure traces recorded from the same cat. Stimulation of S2 ventral root induced colon contractions >20 cmH₂O at frequencies 7-30 Hz with a similar contraction amplitude in the distal and proximal colon (FIG. 3, panel B). Stimulation of the S1 or S3 ventral root did not induce colon contractions larger than the small (<10 cmH₂O) rhythmic baseline colon contractions that were occurring asynchronously during the periods of stimulation (FIG. 3, panels A and C). In this cat, the stimulation intensity used for S2 ventral root stimulation is 2 times the threshold intensity used during 7 Hz stimulation to induce an observable proximal colon contraction. Due to the weak colon response to S1 and S3 ventral root stimulation, the maximal stimulation intensity just below the threshold for inducing lower spine movement was applied to these roots. Hindlimb (mainly S1/S2 stimulation) and/or tail (mainly S2/S3 stimulation) movements were induced by stimulation of each of the three roots. FIG. 3 summarizes the results from 7 cats showing that S2 ventral root stimulation at 7-10 Hz was optimal for producing significantly (p<0.05) larger colon contractions than those elicited by S1 or S3 ventral root stimulation. There was no significant difference between the contraction amplitudes of distal and proximal colon (FIGS. 4A-4B).

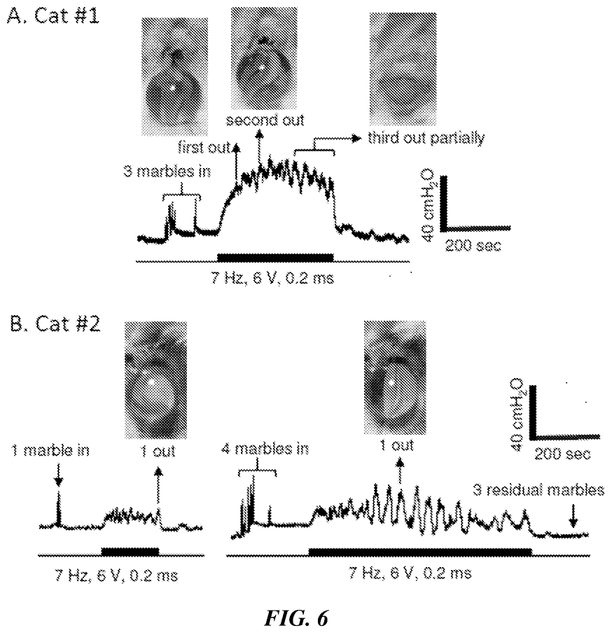

Stimulation (7 Hz) of the S2 ventral and dorsal roots together induced large amplitude (>20 cmH₂O) contractions in both distal and proximal colon (FIG. 5). FIG. 5, panel A shows that intermittent 1-minute stimulation at 1.5 times threshold intensity for inducing a proximal colon contraction produced a stronger contraction during the second period of stimulation and that the large amplitude contraction was maintained throughout the next three 1-minute stimulations. However, the contraction amplitude gradually declined during a continuous 5-minute stimulation, indicating a fatigue in the colon contractions (FIG. 5, panel A). On average the contraction amplitude for both the distal and proximal colon was maintained during the 5 intermittent 1-minute stimulations (N=4 cats, FIG. 5, panel B), but was significantly (p<0.05) reduced from the mean maximal amplitude of 30 cmH₂O to about 18 cmH₂O at the end of the 5-minute continuous stimulation (N=7 cats, FIG. 5, panel C).

Stimulation of the S2 ventral and dorsal roots together induced a maximal amplitude colon contraction of 60 cmH₂O in cat #1 (FIG. 6, panel A) and 40 cmH₂O in cat #2 (FIG. 6, panel B). In cat #1 after 3 marbles were inserted into the rectum, S2 root stimulation (7 Hz, 6 V, 0.2 ms) induced a successful defecation that eliminated the first marble after 1 minute of stimulation and the second marble after 2 minutes of stimulation (FIG. 6, panel A). The elimination of the third marble which started—during the last 2 minutes of the 6-minute stimulation was incomplete (FIG. 6, panel A). In cat #2 after 1 marble was inserted into the rectum, the S2 root stimulation (7 Hz, 6 V, 0.2 ms) eliminated the marble after 2.5 minutes of stimulation (FIG. 6, panel B). However, after 4 marbles were inserted into the rectum, the stimulation eliminated only 1 of the 4 marbles after 4.5 minutes of stimulation with 3 marbles in the rectum at the end of 11 minutes of stimulation (FIG. 6, panel B). Although stimulation of S2 ventral and dorsal roots together only eliminated 1-2 marbles in these two cats, stimulating the S2 ventral root alone without the dorsal root successfully eliminated all 4 marbles in both cats.

### Discussion

This study in anesthetized cats shows that the colon response to sacral root stimulation is dependent on stimulation frequency and the spinal segment stimulated. S2 ventral root stimulation at 7 Hz is optimal to induce both proximal and distal colon contractions (FIGS. 3-4B). Stimulation of the S2 ventral and dorsal roots together is also effective in inducing colon contraction (FIG. 5, panel A), and intermittent stimulation (1 minute on and 1 minute off) is more fatigue resistant than continuous stimulation (FIG. 5, panels B and C). More importantly, defecation can be induced by S2 spinal root stimulation (FIG. 6).

These results have significant implications for developing a new neuromodulation device to restore defecation function in humans after SCI. Since stimulation of the entire S2 spinal root (ventral and dorsal) is as effective as stimulation of the ventral root alone (FIGS. 4A-4B) and successfully induces defection (FIG. 5), sacral posterior rhizotomy may not be necessary and stimulation of sacral spinal roots instead of ventral roots could be effective in restoring defection function after SCI in human. The major reason to perform sacral posterior rhizotomy is to eliminate the detrusor sphincter dyssynergia (DSD) that can generate high pressure in the bladder and cause kidney damage. However, a high pressure in colon and/or rectum might not be as harmful as the high bladder pressure that can cause kidney failure. If colo-rectal pressure is high enough to overcome the dyssynergic anal sphincter contraction, feces should be evacuated. In addition, recent studies in cats have developed an effective method to transiently suppress sphincter activity by blocking pudendal nerve conduction using kHz electrical stimulation. This method is effective in treating DSD in spinal cord injured cats and could also be applied to treat dyssynergic defecation if a strong enough colo-rectal contraction could not be generated to overcome the dyssynergic anal sphincter contraction. Therefore, a novel neuromodulation device may stimulate the sacral spinal root instead of the ventral root and at the same time prevent DSD and/or dyssynergic defection by blocking pudendal nerve conduction to restore bladder function as well as defection function after SCI. The significance of stimulating the sacral spinal root instead of the ventral root lies in the potential to employ a minimally invasive surgical approach for stimulation. S1-S3 spinal roots are accessible by inserting a foramen needle percutaneously to deploy a stimulating lead electrode. This surgical approach has been routinely used in sacral neuromodulation therapy for overactive bladder.

S2 ventral root stimulation eliminated all 4 marbles, but stimulation of S2 ventral and dorsal roots together only eliminated 1-2 marbles (FIG. 6). This difference suggests that stimulating sensory nerves in the dorsal root might have induced additional reflexes to the anal sphincter, which caused dyssynergic defecation and prevented complete emptying of the rectum. This possibility was shown clearly in cat #1 where the evacuation of the third marble was incomplete due to the anal sphincter contraction holding the marble in place (FIG. 6, panel A). If the pudendal nerve were blocked in this experiment, the external anal sphincter would have been relaxed and the third marble might be fully evacuated. However, pudendal nerve block will be ineffective if the dyssynergic defecation is caused by the contractions of internal anal sphincter (smooth muscle) instead of the external anal sphincter (striated muscle). The internal anal sphincter and rectal contraction pressures were not recorded in this study in order to insert marbles into the rectum to test defecation. A small movable balloon in the distal bowel is needed to measure pressure simultaneously as the balloon is eliminated during defecation. Thus, additional studies are warranted to better understand the rectum and anal sphincter coordination during the defecation induced by sacral root stimulation.

In this study the colon contraction and defection induced by S2 root stimulation always required a stimulation intensity higher than the intensity to induce hindlimb and/or tail movement. This result is expected because in cats the afferent and efferent nerve fibers in the S2 spinal root innervating the colon are small unmyelinated C-fibers that have higher excitation threshold than the large motor fibers. Since small C-fibers in the dorsal root are also involved in transmission of nociception, the stimulation intensity required for defection could also generate painful sensation at the same time. This should not be a critical issue for restoring defecation function in people with a complete SCI since they have no sensation below the level of the injury. However, it will be a problem to induce defecation for non-SCI people with chronic constipation if the extrinsic innervation of the human colon is similar to that in cats.

Recent application of sacral neuromodulation to treat non-SCI people with chronic constipation has generated inconclusive clinical outcome, which could be partially due to the use of a low stimulation intensity that only produces somatic sensations. This low stimulation intensity is probably not directly stimulating the nerve fibers in the sacral spinal root innervating the colon. However, it may modulate colon function indirectly by activating the large somatosensory fibers in the sacral root that can trigger central mechanisms to facilitate colon motility. This central modulation mechanism could be sensitive to stimulation frequency, but only a single stimulation frequency of 14 Hz was used in recent clinical trials to treat non-SCI people with chronic constipation. Our study shows that a lower frequency 7 Hz is optimal for sacral root stimulation to induce colon contractions, while other studies in rats and dogs found an effective frequency of 5 Hz or 10 Hz. Therefore, it seems that a frequency lower than 14 Hz should be tested in clinical studies to treat non-SCI people with chronic constipation.

Our study also shows that continuous stimulation can cause fatigue of colon contractions while intermittent stimulation is more fatigue resistant (FIG. 5). This indicates that the continuous stimulation of sacral spinal root used in recent clinical trials to treat chronic constipation may not be optimal and intermittent stimulation may be more effective in modulating colon motility to improve colon transit time. Recent animal studies have begun to focus on developing optimal stimulation parameters to facilitate colon motility and improve sacral neuromodulation therapy for non-SCI people with chronic constipation.

In summary, this study in cats optimized the stimulation parameters for sacral spinal root stimulation to induce colon contraction and defection. The results have significant implications for design of a novel neuromodulation device to restore defecation function after SCI, and for optimizing sacral neuromodulation parameters to treat non-SCI people with chronic constipation.

### EXAMPLE 2

The experimental protocol and animal use in this study were approved by the Animal Care and Use Committee at the University of Pittsburgh.

### Materials and Methods

A total of 8 male cats (4.6±0.2 kg, domestic shorthair) were anesthetized with isoflurane (2-5% in oxygen) during surgery and then switched to α-chloralose anesthesia (initial 65 mg/kg i.v. and supplemented as needed) during data collection. Left cephalic vein was catheterized for fluid administration. A tracheotomy was performed, and a tube was inserted to keep the airway patent. A catheter was inserted into right carotid artery to monitor systemic blood pressure via a pressure transducer (BLPR2 WPI, Sarasota, FL) connected to an amplifier (TBM4M, WPI, Sarasota, FL). Heart rate and blood oxygen were monitored by a pulse oximeter (9847V; NONIN Medical, Plymouth, MN) attached to the tongue. Through an abdominal incision, a catheter was inserted into the bladder via urethra to drain the bladder during the experiment and the urethra was tied with a suture. Then, the abdominal incision was closed with sutures.

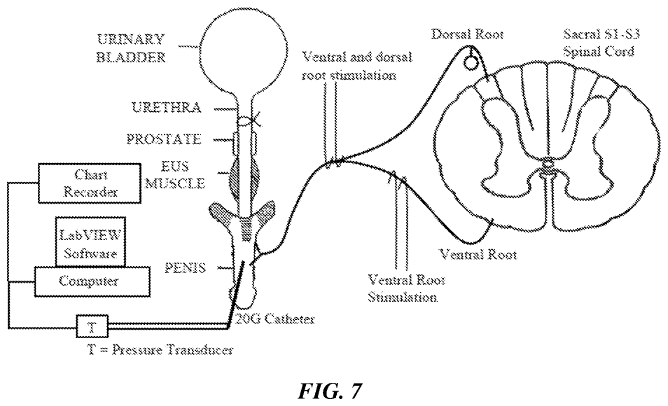

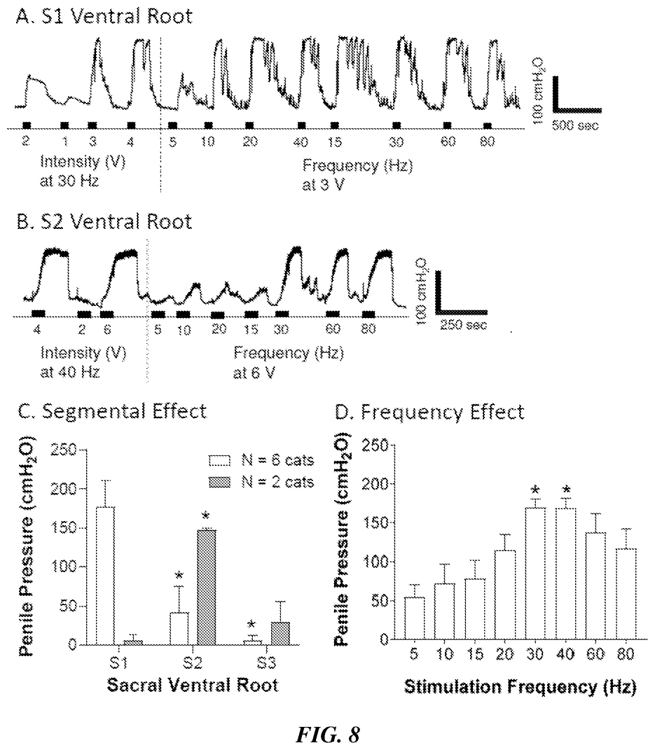

The spinal cord was exposed from lumbar L7 to sacral S3 segments by a dorsal laminectomy. The dura mater was opened and each of the S1-S3 roots was identified. The dorsal and ventral roots were separated to either stimulate the ventral root individually or to stimulate both dorsal and ventral roots together (FIG. 7). The animals were in a prone position during the experiment with a surgical retractor maintaining the spinal incision open to form a pool that was filled with warm (35-37° C.) mineral oil. A heating pad was used to maintain the animal body temperature between 35 and 37° C. Bipolar stainless steel hook electrodes (2-3 mm distance between the electrodes) were used to deliver the stimulation to each sacral spinal root by slightly lifting the root above the spinal cord. The stimulation was generated by a stimulator (S88, Grass Instruments, West Warwick, RI) and delivered via a constant voltage stimulus isolator (SIU5A, Grass Instruments, West Warwick, RI).

At the beginning of the experiment, stimulation (30 Hz frequency, 0.2 ms pulse width, and 1-2 min duration) was delivered via the hook electrode to sacral S1 ventral root at a minimal intensity (0.5 V). If no erection was observed, the stimulation intensity was increased and then tested again until a penile erection was observed. If S1 ventral root stimulation was not effective, then the stimulation electrode was moved to the S2 ventral root, and the test was repeated. A penile erection, which was evident as a full protrusion of the penis with rigidity resistant to bending, was always observed by stimulation of the individual S1 and S2 ventral roots. After the penile erection was observed, a 20-gauge catheter was inserted into the corpus cavernosum through a small incision at the tip of the penis to record penile pressure during the erection (FIG. 7) via a pressure transducer (BLPR2 WPI, Sarasota, FL) connected to an amplifier (TBM4M, WPI, Sarasota, FL). The pressure data were recorded on a chart recorder (TA4000, Gould, Chandler, AZ) and digitized by a computer running a LabView program (National Instruments, Austin, TX). Since electrically evoked penile protrusion is very well correlated with increased corpus cavernosum pressure, the pressure recording was used as a quantitative measurement of penile erection in this study.

After placement of the penile catheter (N=8 cats), stimulation (30 or 40 Hz frequency, 0.2 ms pulse width, and 1 min duration) was delivered via the hook electrode to individual left or right sacral ventral roots (S1, S2, or S3) at a range of intensities (0.5-15 V) to determine the intensity threshold (T) for inducing an observable increase in penile pressure. Then, an intensity (1.5-3T) that induced the maximal penile pressure (>100 cmH₂O) was used to test penile responses to different stimulation frequencies (5-80 Hz). If no penile pressure >100 cmH₂O was induced by stimulation of an individual ventral root, the maximal stimulation intensity that did not produce movement of the animal's lower body was used to test the frequency response. It was intended not to fix the hip and lower spine, so that sacral root stimulation by the hook electrode could produce local movement of the tail or hindlimb. However, the maximal stimulation intensity was limited by any movement of the lower spine, which would displace the hook electrode from the nerve. After testing each ventral root, the ventral root (S1 or S2) that elicited the largest penile pressure was combined with the dorsal root and the combination was stimulated continuously for 10 mins with the most effective frequency (30 Hz) and intensity to determine if the induced penile erection was sustainable during the entire stimulation period. Finally, the spinal cord was completely transected at the T9-T10 level (N=6 cats). About 10 minutes after the spinal cord transection, the continuous 10-minute stimulation of the same spinal root (ventral and dorsal roots together) was tested again to induce penile erection. No drug was used to treat the blood pressure change caused by the spinal transection.

To compare the penile erection responses induced by stimulation of different ventral roots at different frequencies, the maximal amplitudes of the penile pressure induced by stimulation were measured and averaged across different animals for the same stimulation conditions. To determine the sustainability of the induced penile election, the maximal penile pressure induced by the continuous 10-minute stimulations was compared to the pressure at the end of the 10-minute stimulation. To determine the effect of the spinal cord transection, the maximal penile pressures induced by the continuous 10-minute stimulation were compared before and after the spinal cord transection. The data from different animals are averaged and presented as means±SE. Paired t-test or repeated-measures Friedman test followed by Dunnett's multiple comparison were performed for statistical analysis using a software package (Prism 9.3.1, GraphPad Software, San Diego, CA). The statistical significance was defined as p<0.05.Results

Penile erection was observed as a full protrusion of the penis with rigidity in 6 cats by stimulation of S1 ventral root (S2 and S3 ineffective) and in 2 cats by stimulation of S2 ventral root (S1 and S3 ineffective). The stimulation parameters were 30 Hz frequency, 0.2 ms pulse width, and intensity 4.3±1.0 V (0.5-8 V). These responses which were quantified by penile pressure recordings showed that S1 or S2 ventral root stimulation in 6 cats and 2 cats, respectively, induced a large increase in penile pressure from the baseline pressure of 25±1 cmH₂O to 177±14 cmH₂O (S1) or 147±2 cmH₂O (S2) (FIG. 8, panel C). The optimal stimulation frequency to induce penile erection by S1 or S2 ventral root stimulation was 30-40 Hz (FIG. 8, panel D), although lower frequencies (10-20 Hz, FIG. 8, panel A) or higher frequencies (60-80 Hz, FIG. 8, panels A and B) could also induce large increases in penile pressure in some cats. The stimulation intensity for 30-40 Hz S1 or S2 ventral root stimulation to induce the largest increase in penile pressure was 3.5±1.4 V (0.6-12 V) that was always above the motor threshold to induce leg muscle contraction (S1) or anal sphincter contraction (S2).

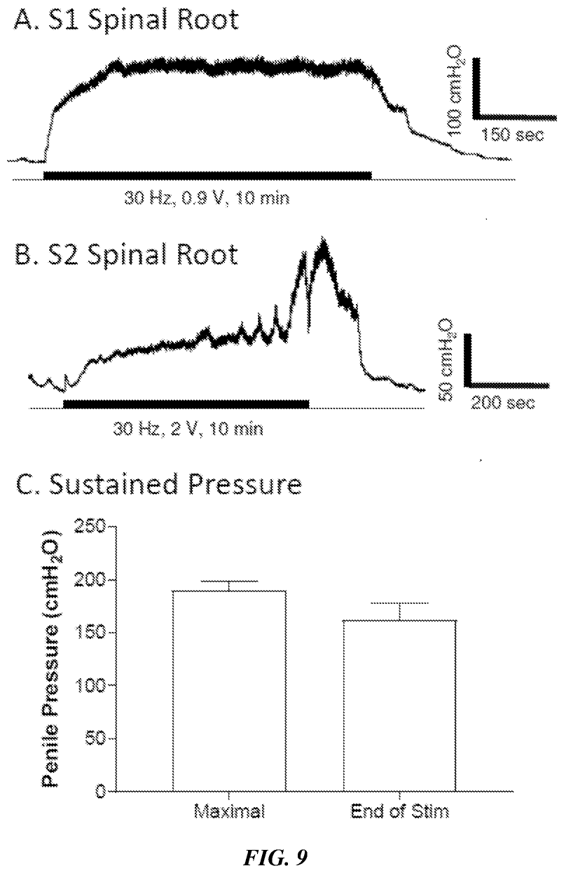

Continuous 10-minute stimulation (30 Hz) of the S1 or S2 spinal root (i.e., ventral and dorsal roots together) induced a large increase in penile pressure (190±8 cmH₂O) (FIG. 9). The maximal penile pressure was either sustained (FIG. 9, panel A) or gradually developed (FIG. 9, panel B) during the 10-minute stimulation. The penile pressure at the end of 10-minute stimulation was not significantly different from the maximal pressure developed during the stimulation (FIG. 9, panel C). The maximal penile pressure induced by stimulation of ventral and dorsal root together was also not significantly different from the maximal pressure induced by stimulation of a ventral root alone (FIG. 8, panel D and FIG. 9, panel C).

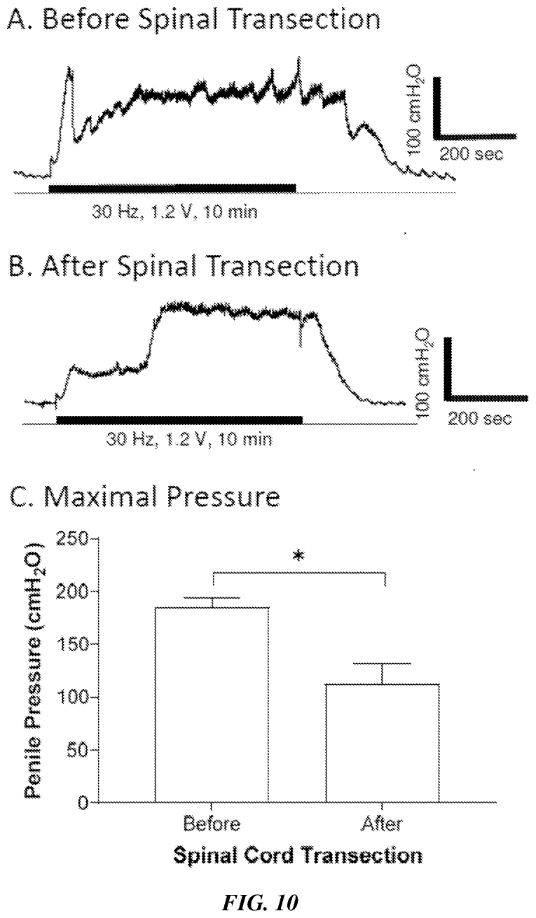

After a complete spinal cord transection at the T9-T10 level, continuous 10-minute stimulation (30 Hz) of the S1 or S2 spinal root (ventral and dorsal roots together) also induced a large increase (186±9 cmH₂O) in penile pressure (FIG. 10). As seen prior to spinal transection, the maximal penile pressure was also sustained or gradually developed (FIG. 10, panel B) during the 10-minute stimulation. However, the induced penile pressure was significantly (p=0.0025) reduced to 113±19 cmH₂O after spinal cord transection when compared to the penile response (186±9 cmH₂O) before the spinal cord transection (FIG. 10, panel C). The blood pressure was also significantly (p=0.0027, N=6) reduced from 229±11 cmH₂O to 186±7 cmH₂O by spinal cord transection, but it was not changed by sacral root stimulation.

### Discussion

This study in anesthetized cats shows that the penile erection induced by sacral root stimulation is dependent on stimulation frequency and the spinal segment stimulated. In each animal one spinal root, most often S1, which contains a minor component of the parasympathetic efferent outflow to the pelvic viscera in the cat, elicited the most prominent response. The largest increases in penile pressure occurred at 30-40 Hz stimulation (FIG. 8), were sustainable during prolonged (10 min) stimulation (FIG. 9) and persisted after an acute complete transection of the spinal cord (FIG. 10). These data suggest that the efficacy of sacral neuromodulation in improving penile erectile function in people with spinal cord injury will depend on the selection of the appropriate spinal root and the optimal stimulation frequency.

Previous studies have shown that penile erection can be induced by electrical stimulation of pelvic nerve, cavernous nerve, or dorsal penile nerve in mice, rats, cats, dogs, monkeys, or humans. However, the pelvic and cavernous nerves require invasive surgery for implantation of stimulation electrodes while the dorsal penile nerve is not a convenient location to attach a stimulation electrode during sexual intercourse. Therefore, these stimulation methods are not broadly adopted for clinical application to treat erectile dysfunction. Sacral ventral root stimulation was also shown to be effective in inducing penile erection in this study and in previous studies using cats and dogs. In addition, sacral anterior root stimulation was used to restore bladder function and erectile function for some SCI people. However, invasive spinal surgery is required to implant the stimulation electrodes on the sacral anterior roots, preventing this effective treatment to be used in SCI or non-SCI people for the sole purpose of treating erectile dysfunction. This study in cats indicates that penile erection can be induced by stimulation of the sacral spinal roots. A previous study in dogs also showed a similar effect. The significance of stimulating the sacral spinal root instead of the ventral root lies in the potential to employ a minimally invasive surgical approach for stimulation. Sacral spinal roots in humans are accessible by inserting a foramen needle percutaneously to deploy a stimulating lead electrode. This surgical approach has been routinely used in sacral neuromodulation therapy for overactive bladder. Therefore, the present study has significant implications for developing a new sacral neuromodulation therapy using a minimally invasive surgical approach to restore erectile function for both SCI and non-SCI people.

Previous studies using sacral neuromodulation to treat overactive bladder also evaluated the effects on penile erection in both SCI and non-SCI people. Questionnaires indicate that subjects with moderate-mild erectile dysfunction significantly improved their quality of sexual life by sacral neuromodulation therapy. However, these studies used stimulation parameters optimized to treat overactive bladder, i.e., stimulation frequency 20 Hz at the sensory threshold intensity. These stimulation parameters may not be optimal for the treatment of erectile dysfunction because the study indicates that a higher stimulation frequency (30-40 Hz) at intensities above sensory and motor thresholds can induce penile erection quickly once the stimulation is turned on and the erection can be sustained during the entire 10-minute stimulation. This type of on-demand erection should be much more satisfactory for the patient than the quality-of-life improvement produced by continuous (24 hours×7 days) sacral neuromodulation at a lower frequency and weaker stimulation intensity. Therefore, it seems that a higher frequency (30-40 Hz) and a stronger stimulation intensity (above sensory and motor thresholds) should be tested in clinical studies to treat erectile dysfunction. Although leg movement will be induced by a stronger stimulation, the leg muscle contraction will be tonic and the movement will only occur at the beginning of the stimulation, which should not be a problem for successful sexual intercourse. When anterior root stimulation was used to induce penile erection in SCI people, it also induced leg movement but did not prevent successful sexual intercourse.

Stimulation of the sacral spinal root (ventral and dorsal roots together) induced a large increase in penile pressure similar to that produced by stimulation of the ventral root alone (FIG. 8). This result indicates that activation of additional sensory nerves in the dorsal root did not disrupt the penile erection induced by direct stimulation of the efferent nerves in the ventral root. A previous study in dogs also reported no difference in penile erection elicited by ventral root stimulation alone or by stimulation of ventral and dorsal roots together. The threshold intensity for inducing leg movement or anal sphincter contraction was not measured in this study, which makes it difficult to estimate what types of nerve fibers were activated in the sacral roots when penile erection was induced. In human application under awake conditions, activation of sensory nerves may cause uncomfortable or painful sensation that can limit the stimulation intensity and therefore limiting the efficacy of the stimulation in treating erectile dysfunction. This potential issue can only be resolved by a clinical study in non-SCI human subjects. However, it should not be an issue for subjects with a complete SCI who have no sensation below the injury.

This study shows that the penile erection induced by sacral root stimulation is sustainable or gradually develops during the 10-minute stimulation (FIG. 9, panels A-B). This indicates that sacral root stimulation can produce long duration erections when it is applied for a longer time. It also indicates that stimulation of afferent nerves in the dorsal root did not reflexively activate the inhibitory sympathetic pathway that can suppress penile erection. Sacral root stimulation probably activated both parasympathetic and somatic pathways to produce the erection and penile rigidity. However, after spinal cord transection the amplitude of the induced penile pressure was significantly reduced, possibly due to the spinal shock effect and the decrease in blood pressure after an acute spinal cord transection. Nevertheless, sacral root stimulation still induced a large (>100 cmH₂O, FIG. 10) sustained increase in penile pressure after acute spinal cord transection, indicating that this stimulation could be useful in restoring erectile function after SCI. Whether sacral root stimulation will be less effective in SCI people than in non-SCI people to induce penile erection is a question to be answered by human clinical studies. However, it is known that sacral anterior root stimulation is effective in inducing penile erection strong enough for sexual intercourse in SCI people. In summary, this study in cats optimized the stimulation parameters for sacral spinal root stimulation to induce penile erection. The results have significant implications for design of a novel neuromodulation device to restore erectile function after SCI, and for optimizing sacral neuromodulation parameters to treat non-SCI people with erectile dysfunction.

While the present invention has been described in terms of the above detailed description, those of ordinary skill in the art will understand that alterations may be made within the spirit of the invention. Accordingly, the above should not be considered limiting, and the scope of the invention is defined by the appended claims.

1. A method of inducing colon contractions and/or defecation in a patient, comprising stimulating one or more sacral roots of the patient's spinal cord and/or one or more sacral segments of the patient's spinal cord with a plurality of electrical pulses, wherein the electrical pulses are delivered at a frequency of from about 3 Hz to about 10 Hz.

2. The method of claim 1, wherein the one or more sacral roots or sacral cord segments are one or more of the patient's S1, S2, S3, S4, and/or S5 sacral roots or sacral cord segments.

3-6. (canceled)

7. The method of claim 1, wherein the stimulation is applied to the patient's S2 and/or S3 ventral/anterior root.

8. The method of any of claim 1, wherein the stimulation is applied at a frequency of about 7 Hz, optionally at an intensity ranging about 0.1 V to about 20 V and/or about 0.1 mA to about 20 mA.

9. (canceled)

10. The method of claim 8, wherein the stimulation is applied at an intensity of about 1 V and/or 1 mA.

11. The method of claim 8, wherein the stimulation is applied at an intensity of about 4 V and/or 4 mA.

12. The method of claim 8, wherein the stimulation is applied at an intensity of about 6 V and/or 6 mA.

13. The method of claim 1, wherein the stimulation is applied continuously or intermittently.

14. The method of claim 13, wherein the intermittent stimulation is applied for about 1 minute followed by about 1 minute where no stimulation is applied.

15. A method of inducing a penile erection in a patient, comprising stimulating one or more sacral roots of the patient's spinal cord and/or one or more sacral segments of the patient's spinal cord with a plurality of electrical pulses, wherein the electrical pulses are delivered at a frequency of from about 10 Hz to about 80 Hz.

16. The method of claim 15, wherein the one or more sacral roots or sacral cord segments are one or more of the S1, S2, S3, S4, and/or S5 sacral roots or sacral cord segments.

17-20. (canceled)

21. The method of claim 16, wherein the stimulation is applied to the S1 and/or S2 ventral/anterior root.

22. The method of claim 15, wherein the stimulation is applied at a frequency of about 30 Hz to about 40 Hz, optionally at an intensity ranging about 0.1 V to about 20 V and/or about 0.1 mA to about 20 mA.

23. (canceled)

24. The method of claim 22, wherein the stimulation is applied at an intensity of about 3 V and/or 3 mA.

25. The method of claim 22, wherein the stimulation is applied at an intensity of about 6 V and/or 6 mA.

26. The method of claim 22, wherein the stimulation is applied at an intensity of about 6 V and/or 6 mA.

27. The method of claim 15, wherein the stimulation causes an increase in pressure in the patient's corpus cavernosum of at least 50 cm H₂O.

28. The method of claim 15, wherein the stimulation causes an increase in pressure in the patient's corpus cavernosum of at least 50 cm H₂O.

29. A system comprising:
at least one lead configured to be placed in proximity to one or more sacral roots of the patient's spinal cord and/or one or more sacral segments of the patient's spinal cord;
a pulse generator in electrical communication with the at least one lead; and
at least one processor in communication with the pulse generator, wherein the processor is programmed or configured to cause the pulse generator to deliver one or more electrical pulses through the at least one lead at a frequency of from about 3 Hz to about 10 Hz, or from about 10 Hz to about 80 Hz.

30. The system of claim 29, wherein the processor is programmed or configured to cause the pulse generator to deliver one or more electrical pulses through the at least one lead at a frequency of about 7 Hz, optionally at an intensity ranging about 0.1 V to about 20 V and/or about 0.1 mA to about 20 mA.

31-37. (canceled)

38. The system of claim 29, wherein the processor is programmed or configured to cause the pulse generator to deliver one or more electrical pulses through the at least one lead at a frequency of about 30 Hz to about 40 Hz, optionally at an intensity ranging about 0.1 V to about 20 V and/or about 0.1 mA to about 20 mA.

39-42. (canceled)
